(19) United States
(12) Patent Application Publication
(10) Pub. No.: US 2026/0069737 A1
(43) Pub. Date: Mar. 12, 2026

(54) Scent Release System, Scent Device, Scent Carrier and Method for Dispensing Scents in Extended Reality Applications

(71) Applicant: Telefonaktiebolaget LM Ericsson (publ), Stockholm (SE)

(72) Inventors: Tobias Widmark, Lund (SE); Andreas Kristensson, Södra Sandby (SE)

(21) Appl. No.: 19/108,553

(22) PCT Filed: Sep. 9, 2022
(86) PCT No.: PCT/EP2022/075100

§ 371 Date: Mar. 4, 2025

(51) Int. Cl. A61L 9/12; A63F 13/28; G06F 3/01

(52) U.S. Cl. CPC A61L 9/125; A61L 9/122; A63F 13/28; G06F 3/011; A61L 2209/134

(57) ABSTRACT

A scent release system (100) for dispensing scents to a user of an XR device (110). The scent release system (100) comprises a scent device (120) a scent carrier (130). The scent device (120) comprises a signal processing unit (121) configured to communicate data with the XR device (110); a scent positioning unit (122) configured to select, based on data received from the XR device, a scent area (131, 132 . . . 13n) on a scent carrier (130) comprising one or more micro-encapsulated scent areas (131, 132 . . . 13n) distributed on the surface of the scent carrier (130); and a scent release unit (123) configured to release a scent from the selected scent area.

WO

### TECHNICAL FIELD

Embodiments herein relate to extended reality applications. In particular, they relate to a scent release system, a scent device, a scent carrier and method therein for dispensing scents in extended reality applications.

### BACKGROUND

Virtual Reality (VR) is a term for technology that involves creating a virtual environment that a user can interact with. Today this interaction is often performed via head-mounted displays (HMDs), as they allow a user to view a 3D representation of the virtual space with real-life motions being tracked and mirrored inside the virtual reality. VR can also be represented in other senses, most commonly being spatial audio and haptic feedback within hand controllers.

Augmented Reality (AR) is a term for technology that involves combining the real world with virtual environments, enhancing or changing a user's perception of the real world. Virtual objects are represented by using displays, speakers, haptics, or other mediums, intersecting with the physical real-world environment. An example would be a pair of glasses that has an integrated heads-up display that can show for example a virtual model of a building in a place where another building is already standing. Depending on the application, objects in the real world can influence virtual objects and vice versa.

When referring to VR and AR together, extended reality (XR) or mixed reality (MR) is often used as an umbrella term.

While visual and auditory stimuli have been at the forefront when it comes to XR development, affecting other senses is seen as a key element to improve further immersion in XR space. Using olfactory stimuli or scents to enhance audio-visual experiences has been attempted for a long time. The solutions available today are commonly adapted to fit conventional VR HMDs. Scents can be used to create an ambient olfactory landscape such as a grassy vista, an oily car shop or a pier by the salty ocean, or to smell specific virtual objects that a user is holding such as a rose or piece of chocolate. Users who engage with XR with an olfactory component congruent to their virtual environment report a higher level of immersion and satisfaction, even when the fragrances are light enough not to be consciously noticed.

With VR devices that are available today, the most common use cases for olfactory devices are found in gaming, health and relaxation.

Current technologies use a liquid perfume-based cartridge system, where a block with a limited number of scents is inserted into a device mounted on a VR HMD, and then later releases scents as the user engages with the virtual environment. Due to the cartridges requiring perfume vials for each scent the number of scents is limited to only a few, e.g. 9 scents, that can be utilized at the same time. Since the devices need to fit the cartridges along with the necessary mechanics, they tend to be significantly bulky as well, often being half of an HMD in size, as shown in FIG. 1, where a schematic drawing shows a peripheral device 11, e.g. a liquid perfume-based cartridge, is mounted onto an HMD 12. The peripheral device 11 is the box to the bottom, which is almost half of the HMD 12 in size, attached to the underside of the HMD 12.

There are also options for room-scale olfactory devices that use a similar cartridge system but emit scents into an entire room. These are not marketed for consumers but rather commercial attractions, such as VR room experiences.

Due to the existing solutions requiring vial cartridges, they can be expensive for a consumer to acquire and may be not widely adopted by the existing XR userbase.

Therefore, there is a need to develop new ways of providing olfactory information to users which will open avenues for more cognitive immersion related to XR products, allow for a fuller experience and more users adopting XR technology.

### SUMMARY

An object of embodiments herein is to provide an improved scent release system that overcomes the limitations and disadvantages related to the prior art solutions.

A scent release system according to embodiments herein is developed by using the micro-encapsulation technology to store various scents. The scents are then released by the scent release system using a mechanism controlled by an XR device.

According to one aspect of embodiments herein, the object is achieved by a scent device for dispensing scents to a user of an XR device. The scent device comprises a signal processing unit configured to communicate data with the XR device; a scent positioning unit configured to select, based on data received from the XR device, a scent area on a scent carrier. The scent carrier comprises one or more micro-encapsulated scent areas distributed on the surface of the scent carrier. The scent device further comprises a scent release unit configured to release a scent from the selected scent area.

According to some embodiments herein, the scent release unit may be configured to release a scent from the selected scent area by applying any one of abrasion, vibration or liquid to the surface of the selected scent area on the scent carrier.

According to one aspect of embodiments herein, the object is achieved by a scent carrier comprising an identifier and one or more micro-encapsulated scent areas distributed on the surface of the scent carrier.

According to one aspect of embodiments herein, the object is achieved by a scent release system comprises a scent device and a scent carrier described above.

According to one aspect of embodiments herein, the object is achieved by a Head Mounted Unit comprises a scent release system described above mounted to, or attached to, or integrated in the Head Mounted Unit.

According to one aspect of embodiments herein, the object is achieved by a mobile device comprises a scent release system described above integrated in the mobile device or in a case of the mobile device. The mobile device may comprise any kind of smart watches, bracelets, headsets etc.

According to one aspect of embodiments herein, the object is achieved by a handheld XR device controller comprises a scent release system described above integrated in the handheld XR device controller.

According to one aspect of embodiments herein, the object is achieved by a method performed in a scent device for dispensing scents to a user of an XR device. The scent device obtains information, by a detection unit, about a scent carrier based on an identifier of the scent carrier; receives, by a signal processing unit, data from the XR device; selects, by a scent positioning unit, based on the data received from the XR device, a scent area on the scent carrier comprising one or more micro-encapsulated scent areas distributed on the surface of the scent carrier; and releases embodiments herein, by a scent release unit, a scent from the selected scent area.

The solutions according to embodiments herein provide a new way to add olfactory stimulus to XR experiences. Some advantages of the embodiments herein include but are not limited to:
The scent release system according to embodiments herein is a much more compact system for delivering the scents, as it relies on a scent carrier which has a flat surface containing the fragrances with a simple dispensing system, such as applying abrasion, vibration or liquid to one or more scent areas, as opposed to using liquid scents directly in the existing solutions.
The scent release system according to embodiments herein allows more scents to be included in a smaller package. The scent carrier according to embodiments herein comprises micro-encapsulated areas which may have virtually any shape and size, so more types of scents can be packed tighter together than the solutions using vials.
The scent carrier according to embodiments herein may be branded after specific experiences, franchises, companies, and acting as a peripheral to a certain game or experience to further marketing possibilities.
The scent release system according to embodiments herein enables easier transport of the product. As no liquid container is used in the scent carrier according to embodiments herein, it can be easily delivered via small postage letters, packaged with physical games without risk for leakage, brought onto planes without liquid restrictions etc.
The scent release system according to embodiments herein has long lifetime thanks to the micro-encapsulation technology which provides an extremely long lifetime when not actively used, as the micro-encapsulation technology can keep scents available for several years after creation.
The scent release system according to embodiments herein has a potential to have parts e.g. the scent carrier, made in biodegradable materials, allowing for easier recycling and post-use material handling.

Therefore, embodiments herein provide an improved method and scent release system for dispensing scents to a user of an XR device. Embodiments herein can provide olfactory information to users of XR products and allow for a fuller experience and more users adopting XR technology. Compared to prior art, the scent release system according to embodiments herein has smaller size, less weight, wider range of scents and easier adaptivity to XR productors.

### BRIEF DESCRIPTION OF THE DRAWINGS

Examples of embodiments herein are described in more detail with reference to attached drawings in which:

FIG. 1 is a schematic drawing showing an example scent release system according to prior art;

FIG. 2 is block diagram showing a scent release system according to embodiments herein;

FIG. 3 is block diagram showing a scent device according to embodiments herein;

FIG. 4 is block diagram showing a scent carrier according to embodiments herein;

FIG. 5 (a) is a schematic top view of an example of a scent carrier according to embodiments herein;

FIG. 5 (b) is a side view of a scent disc according to embodiments herein;

FIG. 6 is an example of a communication system in which embodiments herein may be implemented;

FIG. 7 is a flow chart illustrating an example procedure for AR application using virtual scents according to embodiments herein; and

FIG. 8 is a flow chart illustrating a method for dispensing scents to a user of an XR device according to embodiments herein.

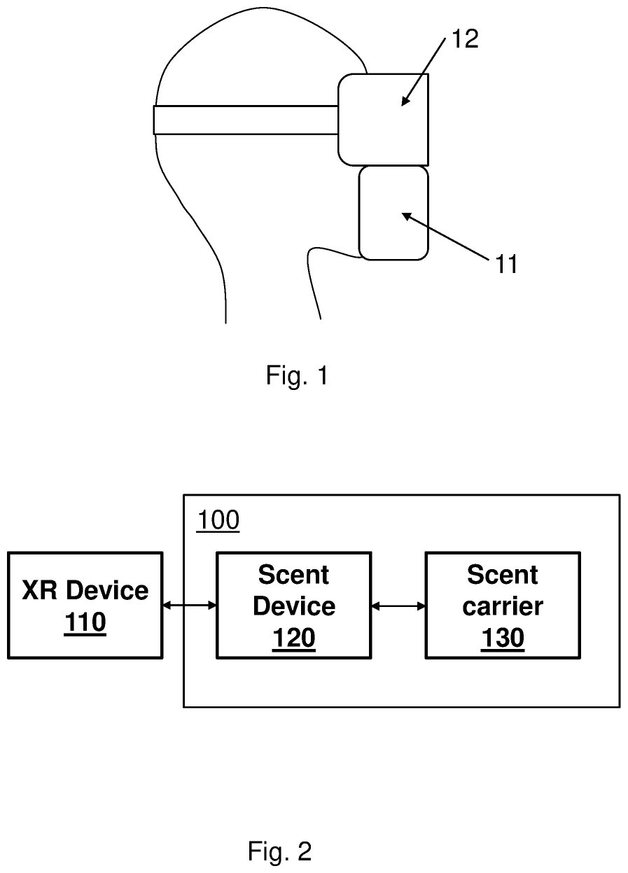

### DETAILED DESCRIPTION

As a part of developing embodiments herein some problems or limitations associated with the existing solutions will be first identified and discussed.

One of the key questions regarding XR technology is how to provide information to users without overloading or overwriting other senses. With olfactory stimuli, this is especially important as our sensitivity to smells makes us susceptible to virtual smells lingering when they should be gone, and if one perceives a scent as incongruent with what he/she is seeing, either by it being the wrong scent or smelling too artificial, he/she may lose immersion instead of enhancing it. The room-scale olfactory devices emit scents into an entire VR room where a lingering smell is not as much of a concern.

The following requirements are missing in the existing solutions:

The olfactory device should be able to support a wider range of scents, to ensure that a larger possibility space is available for the user when exploring virtual environments.

The olfactory device should be light and compact, as any weight added to an HMD translates into additional weight to the user's head and neck resulting in an uncomfortable user experience.

The solution should allow AR devices to use olfactory technology; current solutions are only available for VR devices and would be difficult to adapt due to requiring an HMD that can support the weight.

Micro-encapsulation is a technology of scattering small particles or droplets of a substance onto a surface and then coating it with a membrane, which can later be destroyed to release the substance. The most ubiquitous example related to scent is the “scratch-and-sniff” sticker, a sticker that when scratched emits a smell of some kind, e.g. cola, apple, or motor oil. This toy was popular in the 1980's but has faded in popularity since. Due to the substance being spread out over a surface only part of the sticker needs to be scratched for the scent to release, and the encapsulation means fragrances can remain for several years before showing signs of fading.

A scent release system according to embodiments herein is developed by using the micro-encapsulation technology to store various scents. The scents are then released by the scent release system using a mechanism controlled by an XR device.

FIG. 2 shows a scent release system 100 according to embodiments herein. The scent release system 100 comprises a scent device 120 for dispensing scents to a user of an XR device 110. The XR device 110 may be an AR, VR, or MR device that the user is interfacing with. The scent release system 100 further comprises a scent carrier 130 comprising one or more micro-encapsulated scent areas distributed on the surface of the scent carrier 130.

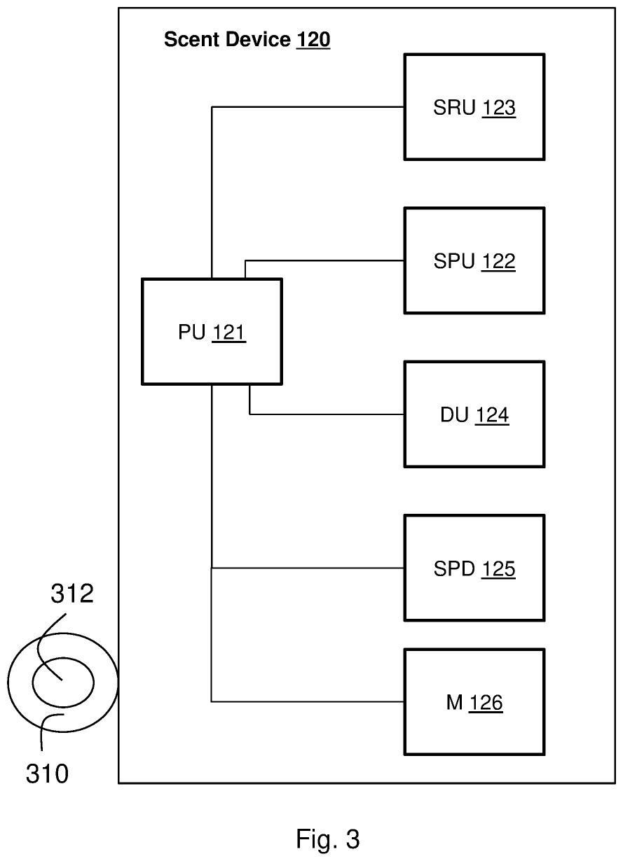

FIG. 3 shows a scent device 120 according to embodiments herein. The scent device 120 comprises a signal processing unit PU 121 configured to communicate data with the XR device 110. When the XR device 110 detects that the user has encountered a virtual object that should emit a simulated scent, it sends a signal containing information or data to the signal processing unit PU 121 to indicate which scent should be released via the scent device 120. The information or data received by the signal processing unit PU 121 may contain the type of scent that should be released, the strength of the scent and the duration the scent should be active. The signal processing unit PU 121 acts as a hub for the data being sent to and from the XR device 110, converting it to commands for the other components of the scent device 120. As certain scents may linger or be more or less strong depending on the type of scent, the signal processing unit PU 121 ensures that the data or values received from the XR device 110 are normalized across the available scents so that they perceived effect is homogenous regardless of the type of scent being released.

The scent device 120 further comprises a scent positioning unit SPU 122 configured to select, based on the data received from the XR device 110, a scent area on the scent carrier 130. The scent carrier 130 comprises multiple micro-encapsulated scent areas distributed on the surface of the scent carrier 130.

The scent device 120 further comprises a scent release unit SRU 123 configured to release a scent from the selected scent area. The scent release unit SRU 123 handles releasing scents from the scent carrier 130. The scent release unit SRU 123 may be configured to release a scent from the selected scent area by applying e.g. abrasion, vibration or liquid to the surface of the selected scent area on the scent carrier 130. That is the scent release unit SRU 123 may use a mechanical solution that applies e.g. abrasion, scratch, scrub, peel, puncture or vibration to the micro-encapsulated scent areas on the scent carrier 130. The mechanical actions may vary in strength and duration, releasing different amounts of scent depending on the information or data received by the signal processing unit PU 121 from the XR device 110. The scent release unit SRU 123 may also drop liquid on the surface of the selected scent area on the scent carrier 130. To release different amounts of scent, the liquid applied may vary in amount.

The scent positioning unit SPU 122 ensures the correct scent is selected and slotted into the scent release unit SRU 123. This may be achieved by using an identifier of the scent carrier 130. The identifier of the scent carrier 130 may provide information on e.g. a shape of the scent carrier 130 and how is the scent carrier 130 mechanically positioned in relation to the scent device 120 so that the scent carrier 130 may be moved or rotated if necessary. For example, if the scent carrier 130 is a disc and due to the shape of the disc, the scent positioning unit SPU 122 may be rotational in nature to easily cycle through the available scent areas. The surface on which the micro-encapsulation is placed may be not a disc, but some other shape like a rectangle or orb. The scent release unit SRU 123 that scratches the surface may be adjusted to function with the alternate shape.

According to some embodiment herein, the scent device 120 may further comprises a detection unit DU 124 configured to obtain information about the scent carrier 140 based on the identifier of the scent carrier 130. The detection unit DU 124 uses the identifier of the scent carrier 130 to get information about which type of the scent carrier 130 is currently used with the scent device 120. The information is passed on to the signal processing unit PU 121 where the scent device 120 can send the types of scents available to the XR device 110. If the application running on the XR device 110 requires certain specific scents, the detection unit DU 124 can ensure the correct scent carrier 130 is being used and inserted, or whether the current scent carrier 130 is able to meet the requirements despite not being the exact correct type of scent carrier.

The detection unit DU 124 may retrieve information about the scent carrier 130 from e.g. the Internet, a cloud or server, or from the scent device 120 based on the identifier of the scent carrier 130. The information about the scent carrier 130 may comprise e.g. a type of the scent carrier 130, e.g. shape of the scent carrier 130 and how is the scent carrier 130 mechanically positioned in relation to the scent device 120, available scents on the scent carrier 130, how much the scents on the scent carrier 130 have been used, how long the remaining lifespan of the scent carrier 130 is left, when the scent carrier 130 needs to be replaced etc.

According to some embodiment herein, the scent device 120 may further comprise a scent propulsion device SPD 125 configured to guide a scent to the user's olfactory system, i.e. to user's nose. The scent propulsion device SPD 125 is to ensure that the scents provided by the scent carrier 130 are properly guided to the user's olfactory system. The specific implementation is outside the scope of this disclosure, but it may be achieved by small fans or guiding tubes as examples. The scent propulsion device SPD 125 may be made more sophisticated by using signals from the signal processing unit 121, such as turning on/off fans depending on how active the scent should be.

The scent device 120 may further comprise a memory M 126 to store information, data, configurations etc.

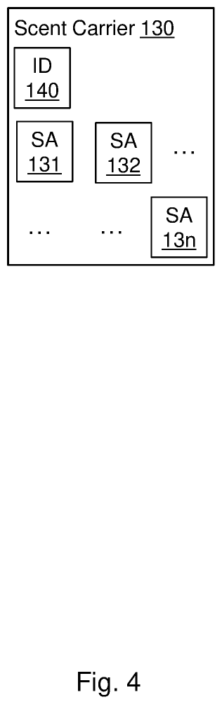

FIG. 4 shows a schematic block view of a scent carrier 130 according to embodiments herein. The scent carrier 130 comprises an identifier ID 140 of the scent carrier 130. The scent carrier 130 further comprises one or more micro-encapsulated scent areas SA 131, 132, . . . 13n distributed on the surface of the scent carrier 130. Each of the scent areas SA 131, 132, . . . 13n is an area that contains a micro-encapsulated scent. The one or more scent areas SA 131, 132, . . . 13n may be distributed on the surface of the scent carrier 130 in a pattern. The scent carrier identifier ID 140 may be a Uniform Resource Locator (URL), a number code, a color combination, a Quick response (QR), code, a near-field communication (NFC), a tag or any other means that can interface with the detection unit 124. The scent carrier identifier ID 140 provides a way for the detection unit 124 to identify the type of scent carrier 130 that the identifier ID 140 is attached to.

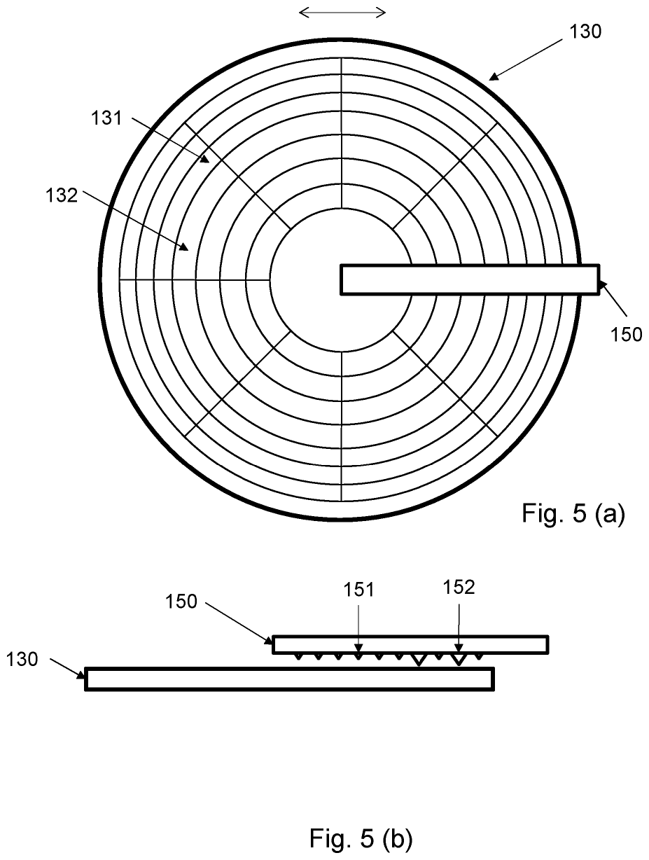

The scent carrier 130 may have any shape, such as a disc, a rectangle or an orb. FIG. 5 (a) is a schematic top view of an example of the scent carrier 130 according to embodiments herein. The scent carrier 130 in this example is a disc-shaped surface with multiple strips of micro-encapsulated areas, e.g. 56 strips of scent areas, indicted by 131, 132, . . . . The scent areas 131, 132 . . . are placed in a pattern around the scent disc's surface. The scent disc 130 may have any size, e.g. 6 cm in diameter. FIG. 5 (b) is a side view of the scent disc 130. A scent release arm 150 is also shown in FIGS. 5 (a) and (b) which is a part of the scent release unit SRU 123. The scent release arm 150 may comprise one or more scent release pins 151, 152, where the scent release pin 151 is an in-active scent release pin and the scent release pin 152 is an active scent release pin.

The scent position unit SPU 122 ensures that the correct scent area 131, 132 . . . is set in position for the scent release unit SRU 123 to release the scent, and the available scents on the scent disc 130 are reflected in the information provided by the identifier of the scent disc 130.

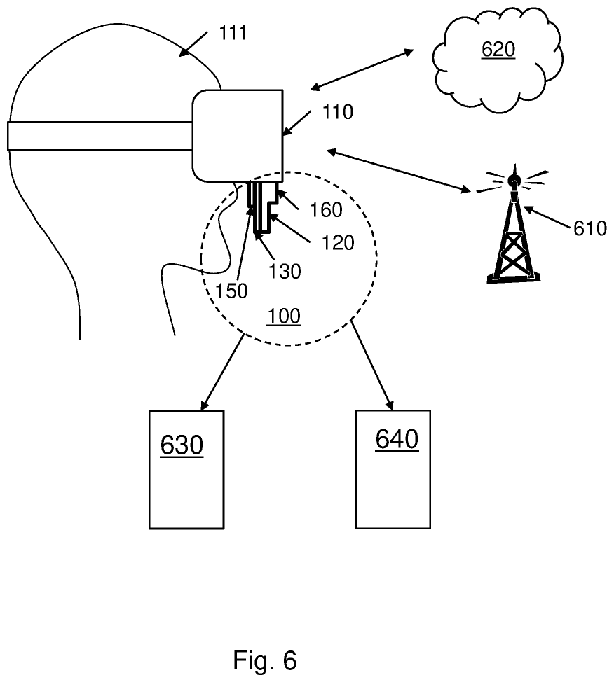

FIG. 6 shows an example of a communication system 600 in which embodiments herein may be implemented. The communication system 600 comprises an XR device 110 associated with a user 111. The communication system 600 further comprises a network node 610, a cloud or server 620. The network node 610 is a network access node which may be a base station (BS), for example, an eNB, gNB, eNodeB, gNodeB, or a Home NodeB, or a Home eNodeB or a Home gNodeB in a wireless communication network. The wireless communication network may be any wireless system or cellular network, such as a second generation (2G) Global System for Mobile Communications (GSM), a third generation (3G) telecommunication network e.g. Universal Mobile Telecommunications System (UMTS), a Fourth Generation (4G) network, e.g. Evolved Packet System (EPS) or Long Term Evolution (LTE) network, a Fifth Generation (5G) New Radio (NR) network, or Wimax network, Wireless Local Area Network (WLAN) or Wi-Fi network etc. The XR device 110 may communicate with the network node 610 and cloud or server 620 to receive and transmit information.

The communication system 600 shown in FIG. 6 is just one example where the XR device 110 is shown as head mounted unit such as head mounted display (HMD) or glasses. The method and device for dispensing scents according to embodiments herein may be applied to any XR devices, such as a mobile device 630 or a handheld XR device controller 640. The mobile device 630 may be any kind of smart watches, bracelets, headsets.

The scent system 100 in this example embodiment comprises a scent device 120 with a supporting armature 160 e.g. motor, electronics etc. for supporting the function of the scent device 120, a scent carrier 130 has a shape of disc, a scent release unit shown as a scent release arm 150. The scent system 100 is mounted onto the HMD 110 as a peripheral device.

The scent device 120 may either be a separate peripheral or integrated into the XR device 110. The scent carrier 130 is a stand-alone item that contains the sets of scents and may be changed for other scent carrier with different sets of scents depending on the needs of the XR application.

According to some embodiments herein, the scent release system 100 may be mounted to, or attached to, or integrated in the head mounted unit 110.

According to some embodiments herein, the scent release system 100 may be integrated in the mobile device 630 or in a case of the mobile device 630.

According to some embodiments herein, the scent release system 100 may be integrated in the handheld XR device controller 640. This will cater for use cases like picking up a virtual flower and holding it close to nose with the handheld XR device controller and then having the scent system 100 in the handheld XR device controller releases a relevant flower smell.

According to some embodiments herein, the scent carrier 130 may be configured to fit a certain software, experience or a number of experiences and made to be replaced with a different scent carrier if another software is used. This may be embodied as the scent carrier 130 is branded after a specific software or type of environment. That is the scent carrier 130 may be adapted to a specific software or application, or a type of environment. The scent carrier 130 may also be adapted to be replaceable by a different scent carrier depending on different software and applications.

According to some embodiments herein, the scent system 110 may be configured to allow several scents to be released at once. For example, the scent release unit SRU 123 can apply abrasion to multiple scent areas and/or the scent positioning unit SPU 122 can reposition the scent carrier fast enough for several scents to blend. This may feasibly be used to combine different scents to create new scents.

Therefore, according to some embodiments herein, the scent release unit SRU 123 may be configured to release two or more scents at once by applying any one of abrasion, vibration or liquid to multiple scent areas. The scent positioning unit SPU 122 may further be configured to select and position scent areas such that several scents are released and blended to create new scents.

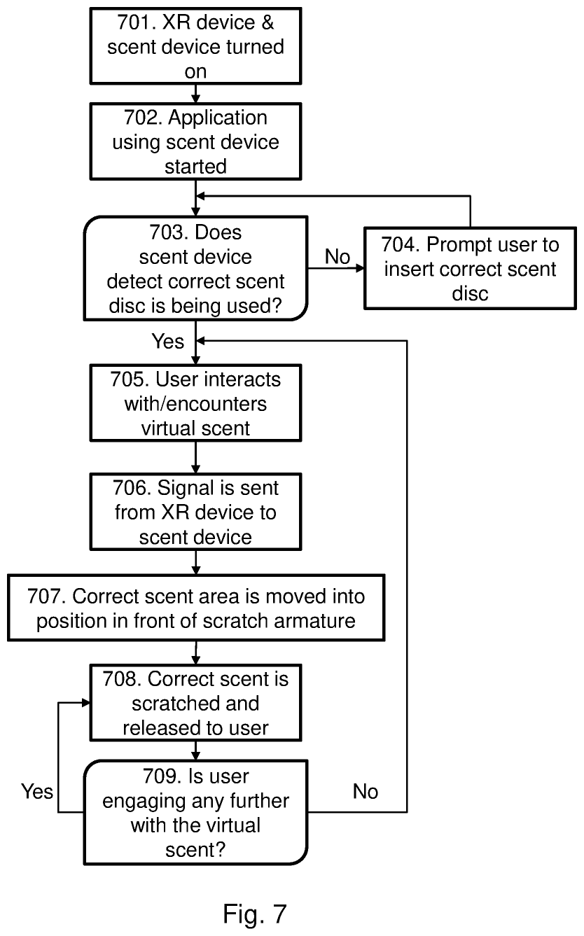

FIG. 7 shows a flow chart 700 showing an example procedure of AR application using virtual scent according to embodiments herein. Although the procedure is described with regard to an example embodiment where the scent carrier 130 is a scent disc and the release unit 123 comprises a scratch armature 150, the procedure may be applied to other types of scent carrier and scent release unit. The procedure flow chart 700 comprises the following Actions.

### Action 701

The XR device 100 and scent device 120 are turned on, simultaneously or separated.

### Action 702

The application using the scent device 120 is started on the XR device 110.

### Action 703

The detection unit DU 124 in the scent device 120 detects whether a correct scent disc 130 regarding to the application is being used based on the scent carrier identifier ID 140 associated with the scent disc 130. That is the AR application requests a scent disc with a specific set of smells that could become relevant during the AR application, prompting a check using the detection unit DU 124 and notifying the user to insert the correct disc if he/she has not already done so. If the application running on the XR device 110 requires certain specific scents, the detection unit DU 124 can ensure the correct scent carrier 130 is being used and inserted, or whether the current scent carrier 130 is able to meet the requirements despite not being the exact correct type of scent carrier.

### Action 704

If the scent disc 130 is not correct, the XR device 110 prompts the user to insert a correct scent disc 130.

### Action 705

In case the correct scent disc 130 is being used, the AR application is running as normal, providing whatever audio-visual experience the XR device 110 is configured for. The user starts interacting with or encountering with virtual scent when the AR application is running.

### Action 706

When a virtual scent becomes relevant, such as when holding up a virtual rose in front of one's nose, the XR device 110 sends a signal for the required scent to the signal processing unit PU 121 in the scent device 120. The signal contains data indicating the type of scent that should be released, e.g. a rose scent, the strength of the scent and the duration the scent should be active.

### Action 707

The signal processing unit PU 121 processes and converts the signal received from the XR device to a command for triggering the scent position unit SPU 122 to rotate or move the scent disc 130 until the correct scent area, e.g. the rose scent, is in the position in front of the user's nose and in the position of the scratch armature 150 of the scent release unit SRU 123 and. That is the scent position unit SPU 122 in the scent device 120 selects and moves the correct scent area into the position of the scent release unit SRU 123.

### Action 708

The scent release unit SRU 123 then applies e.g. abrasion to the surface of the scent area for the duration that is requested by the XR device 110. For example, the scratch armature 150 scratches the scent area and the correct scent is released to the user.

### Action 709

The scent device 120 checks if the user is engaging any further with the current scent, i.e. the rose scent, based on the data received from the XR device 110.

If the user continues to interact with the virtual rose, the scent will be continuously released. So if the user is engaging further with the current scent, the scent device 120 continue to perform Action 708 to scratch the scent area and release the scent to the user.

But if the object, i.e. the rose, is removed, the scent release system 100 goes dormant, awaiting the next virtual scent to release. So if the user is not engaging any further with the current scent, go back to Action 705 and continue the procedure to release the next scent related to the AR application.

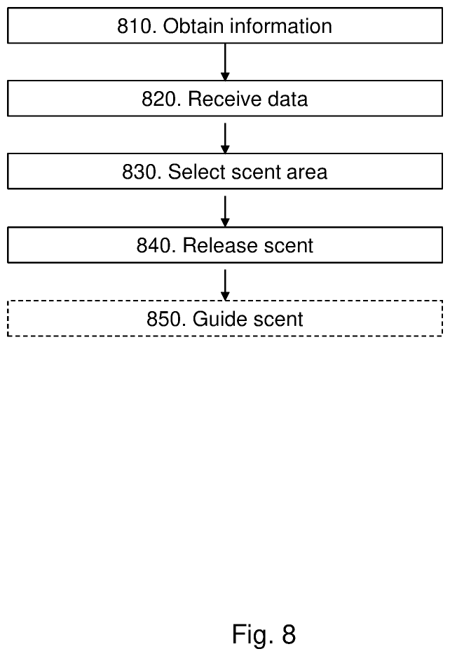

A method performed by the scent device 120 for dispensing scents to a user of the XR device 110 according to embodiments herein will be described with reference to FIG. 8. The method comprises the following actions which may be performed in any suitable order.

### Action 810

To check if the correct scent carrier is being used according to the AR application running in the XR device 110, the scent device 120 obtains, by the detection unit DU 124, information about a scent carrier 130 based on an identifier ID 140 of the scent carrier 130.

The detection unit DU 124 can provide information not only about the type of scent carrier and scents available in the currently used scent carrier, but also about how much the scent carrier has been used, how long its remaining lifespan is left and when it needs to be swapped for a new scent carrier. This may be achieved by having a unique identifier for each scent carrier or a more sophisticated system for the identifier that allows it to keep track of the scent carrier's state outside the scent release system 100. That is the scent release system 100 may store scent related data in e.g. the cloud 620, tied to a specific identifier making it possible for a second system to acquire said data when a partly used scent carrier is inserted into the second system. For example, if a certain scent area has been used a number of times, this information can be stored and retrieved from an online database, instead of from the scent carrier 130 or on the XR device 110.

According to some embodiments herein, the scent device 120 may retrieve information about the scent carrier from the cloud or server 620. This allows the detection unit DU 124 and the scent carrier identifier ID 140 to be very lightweight, as only an URL or number code is the only required data transferred between the scent carrier 130 and the scent device 120.

According to some embodiments herein, the scent carrier identifier ID 140 may be conveyed using color combinations. The detection unit DU 124 can detect the scent carrier type optically, and it also provides a way for the user to get a rough estimate on what kind of scents are available in the scent carrier 130.

### Action 820

The scent device 120 receives, by the signal processing unit 121, data from the XR device 110. The data indicates the type of scent that should be released, the strength of the scent and the duration the scent should be active.

### Action 830

The scent device 120 selects, by the scent positioning unit 222, based on the data received from the XR device 110, a scent area 131, 132 . . . 13n on the scent carrier 130. The scent carrier 130 comprises one or more micro-encapsulated scent areas 131, 132 . . . 13n distributed on the surface of the scent carrier 130.

### Action 840

The scent device 120 releases, by the scent release unit 123, a scent from the selected scent area.

The scent release unit 123 may release a scent from the selected scent area by applying any one of abrasion, vibration or liquid to the surface of the selected scent area on the scent carrier 130.

### Action 850

Optionally, the scent device 120 may guide, by the scent propulsion device 125, the released scent to the user's olfactory system.

Those skilled in the art will appreciate that the signal processing unit PU 121, the scent release unit SRU 123, the scent positioning unit SPU 122, the detection unit DU 124, described above in the scent device 120 may be referred to one circuit or unit, a combination of analog and digital circuits, one or more processors, configured with software and/or firmware and/or any other digital hardware performing the function of each circuit/unit. One or more of these processors, the combination of analog and digital circuits as well as the other digital hardware, may be included in a single application-specific integrated circuitry (ASIC), or several processors and various analog/digital hardware may be distributed among several separate components, whether individually packaged or assembled into a system-on-a-chip (SoC).

The method according to embodiments herein may be implemented through one or more processors in the scent device 120 together with computer program code for performing the functions and actions of the embodiments herein. The program code mentioned above may also be provided as a computer program product, for instance in the form of a data carrier 310 carrying computer program code 312, as shown in FIG. 3, for performing the embodiments herein when being loaded into the scent device 120. One such carrier is shown as the form of a CD ROM disc. It is however feasible with other data carriers such as a memory stick. The computer program code may furthermore be provided as pure program code on a server or cloud 620 and downloaded to the scent device 120.

The memory 126 in the scent device 120 may comprise one or more memory units and may be arranged to be used to store received information, measurements, data, configurations and applications to perform the method herein when being executed in the scent device 120.

It is possible to transfer most of the computational processing to the cloud or server 620, most importantly the signal processing unit PU 121. This may be made more efficient if many of the XR device's features are on the cloud as well, cloud streaming, for example. Some data like the information retrieved from the scent carrier ID 140 may also be retrieved through the cloud or server 620, or from the Internet, as it makes it easier for new scent carries and scent types to be added to the scent release system 100.

To summarize, embodiments herein provide a scent release system 100 for an XR device 110 that uses micro-encapsulation technology to store various scents, that are then released by the scent release system 100 using a mechanism controlled by the XR device 110. That is the scent release system 100 dispenses micro-encapsulated scents based on inputs from the connected XR device 110.

As one example, embodiments herein provide a scent carrier 130 with disc-shaped surface with multiple strips of micro-encapsulated areas containing different kinds of scents, as well as a scent release unit SRU 123 with a mechanical arm 150 comprising one or more pins or other mechanism that can scratch, or scrub, or peel, or puncture those micro-encapsulated scent areas to selectively release the scents depending on the virtual context the user is in.

The scent carrier 130 may contain a system to be recognized by the XR device 110 such as an identifier, a QR code, NFC tag or manual user input from a menu, for identification of what scents the scent carrier 130 can provide.

The scent release system 100 may be positioned in front of the user's nose and may be attached or mounted to, or integrated into, or associated to, or as a part of an XR device 110, e.g. a VR HMD, in such a way that when a signal is sent from the XR device 110, the scent release system 100 rotates or moves the scent carrier 130 until the relevant scent area is positioned in front of the user's nose. The scent release unit SRU 123 then release the fragrance into or near the user's nose by applying abrasion, vibration or liquid to the surface of the selected scent area on the scent carrier 130. For example, the mechanical arm 150 can briefly mechanically scratch the scent area to release the scent.

Embodiments herein also provide a mechanism that enables the scent device 120 to scratch the micro-encapsulated surface, with varying degrees of movement based on the desired type and amount of fragrance needed by an AR application. For example, the scent device 120 may apply abrasion or vibration varying in strength and duration or apply liquid varying in amount to release different amounts of one or more scents based on the received data from the XR device 110.

The scent release system 100 can support a wider range of scents to ensure that a larger possibility space is available for the user when exploring virtual environments.

The scent release system 100 is light and compact which enables comfortable user experience, as any weight added to an HMD translates into additional weight to the user's head and neck resulting in an uncomfortable user experience.

The scent release system 100 allows any AR devices to use olfactory technology. Prior art solutions are only available for VR devices and is difficult to adapt due to requiring an HMD that can support the weight.

When using the word “comprise” or “comprising” it shall be interpreted as non-limiting, i.e. meaning “consist at least of”.

The embodiments herein are not limited to the above described preferred embodiments. Various alternatives, modifications and equivalents may be used. Therefore, the above embodiments should not be taken as limiting the scope of the invention, which is defined by the appended claims.

1-27. (canceled)

28. A scent device for dispensing scents to a user of an Extended Reality (XR) device, the scent device comprising:
a signal processing unit configured to communicate data with the XR device;
a scent positioning unit configured to select, based on data received from the XR device, a scent area on a scent carrier comprising one or more micro-encapsulated scent areas distributed on the surface of the scent carrier; and
a scent release unit configured to release a scent from the selected scent area.

29. The scent device according to claim 28, wherein the scent release unit is configured to release a scent from the selected scent area by applying any one of abrasion, vibration or liquid to the surface of the selected scent area on the scent carrier.

30. The scent device according to claim 28, wherein the scent device further comprises:
a detection unit configured to obtain information about the scent carrier based on an identifier of the scent carrier.

31. The scent device according to claim 30, wherein the information about the scent carrier comprises any one or more of: a type of the scent carrier, available scents on the scent carrier, how much the scents on the scent carrier have been used, a remaining lifespan of the scent carrier, or an indication of when the scent carrier needs to be replaced.

32. The scent device according to claim 30, wherein the detection unit is configured to obtain information about the scent carrier based on the identifier of the scent carrier by being configured to retrieve, based on the identifier, the information about the scent carrier from any the Internet, a cloud, or the scent device.

33. The scent device according to claim 28, wherein the scent device further comprises:
a scent propulsion device configured to guide a scent to the user's olfactory system.

34. The scent device according to claim 33, wherein the scent propulsion device is a fan or a guiding tube.

35. The scent device according to claim 33, wherein the scent propulsion device is configured to turn on or off the fan based on a signal received from the signal processing unit.

36. The scent device according to claim 28, wherein the data received from the XR device comprises one or more of a scent type, a scent strength, and a scent duration.

37. The scent device according to claim 28, wherein the scent release unit is configured to release different amounts of one or more scents based on the received data from the XR device by applying abrasion or vibration varying in strength and duration or by applying liquid varying in amount.

38. The scent device according to claim 28, wherein the scent positioning unit is further configured to select a scent area on the scent carrier based on information on a shape of the scent carrier and how is the scent carrier mechanically positioned in relation to the scent device.

39. The scent device according to claim 28, wherein the scent release unit is configured to release two or more scents at once by applying any one of abrasion, vibration or liquid to multiple scent areas.

40. The scent device according to claim 28, wherein the scent positioning unit is further configured to select and position scent areas such that several scents are released and blended to create new scents.

41. The scent device according to claim 28, wherein the scent release unit comprises a scent release arm comprising one or more scent release pins.

42. The scent device according to claim 28, wherein the scent device further comprises a supporting armature.

43. A scent carrier comprising an identifier and one or more micro-encapsulated scent areas distributed on the surface of the scent carrier.

44. The scent carrier according to claim 43, wherein the scent carrier has any shape of a disc, a rectangle or an orb.

45. The scent carrier according to claim 43, wherein the identifier is any one of a Uniform Resource Locator (URL), a number code, a color combination, a Quick response (QR) code, or a near-field communication (NFC) tag.

46. The scent carrier according to claim 43, wherein the scent carrier is configured to a specific software or application, or a type of environment.

47. The scent carrier according to claim 43, wherein the scent carrier is configured to be replaceable by a different scent carrier depending on different software and applications.

48. A scent release system comprising:
a scent carrier comprising one or more micro-encapsulated scent areas distributed on a surface of the scent carrier; and
a scent device for dispensing scents to a user of an Extended Reality (XR) device, the scent device including: a signal processing unit configured to communicate data with the XR device; a scent positioning unit configured to select, based on data received from the XR device, a scent area on the scent carrier; and a scent release unit configured to release a scent from the selected scent area.

49. The scent release system of claim 48, wherein the scent release system is integrated into one of a head mounted unit, a handheld extended reality (XR) controller, or a mobile communication device.

50. A method for dispensing scents to a user of an Extended Reality (XR) device, the method comprising:
obtaining information, by a detection unit, about a scent carrier based on an identifier of the scent carrier;
receiving, by a signal processing unit, data from the XR device;
selecting, by a scent positioning unit, based on the data received from the XR device, a scent area on the scent carrier comprising one or more micro-encapsulated scent areas distributed on the surface of the scent carrier; and
releasing, by a scent release unit, a scent from the selected scent area.

51. The method according to claim 50, wherein releasing the scent from the selected scent area comprises applying any one of abrasion, vibration or liquid to the surface of the selected scent area on the scent carrier.
